SEQUENCE LISTING (2 sequences)

SEQ ID NO: 1 - protein, 4 aa, Artificial
  consensus sequence for proconvertase
RXXR

SEQ ID NO: 2 - protein, 7 aa, artificial
  sequence including furin-like proconvertase site
RGRRRAR

(19) United States
(12) Patent Application Publication
(10) Pub. No.: US 2010/0266707 A1
(43) Pub. Date: Oct. 21, 2010

(54) METHOD OF TREATING CACHEXIA WITH THE REMOVAL OR INACTIVATION OF MACROPHAGE INHIBITORY CYTOKINE-1

(76) Inventor: Samuel Norbert Breit, New South Wales (AU)

Correspondence Address: NIXON & VANDERHYE, PC, 901 NORTH GLEBE ROAD, 11TH FLOOR, ARLINGTON, VA, 22203, US

(21) Appl. No.: 12/734,098

(22) PCT Filed: Oct. 9, 2008
(86) PCT No.: PCT/AU2008/001498

§ 371 Date: Jun. 22, 2010

(30) Foreign Application Priority Data
Oct. 9, 2007 (AU) 2007905524

(51) Int. Cl. A61K 35/14; A61K 35/16; G01N 33/00; A61P 13/12; A61P 29/00; A61P 35/00

(52) U.S. Cl. 424/529; 424/530; 424/531; 436/86

(57) ABSTRACT

A method of treating cachexia is disclosed involving the removal or inactivation of macrophage inhibitory cytokine-1 (MIC-1) present in the blood, plasma or serum of a cachexia subject. In one embodiment, the method comprises the steps of providing a suitable substrate for binding MIC-1 (e.g. a substrate provided with a MIC-1 binding molecule), treating blood, plasma or serum removed from a subject by contacting the blood, plasma or serum ex vivo with the substrate such that MIC-1 present in the blood, plasma or serum is bound to the substrate, separating the treated blood, plasma or serum from the substrate, and thereafter returning the treated blood, plasma or serum to the subject. Also disclosed, is a method of diagnosing or prognosing cachexia in a subject, said method comprising determining the amount of MIC-1 present in the subject.

### INCORPORATION BY REFERENCE

This patent application claims priority from:
AU 2007905524 titled “A method of treating cachexia” filed on 9 Oct. 2007.

The entire content of this application is hereby incorporated by reference.

Also, the following patent specification is referred to in the following description:
International Patent Application No PCT/AU2005/000525 (WO 2005/099746). The entire content of this specification is hereby incorporated by reference.

### FIELD OF THE INVENTION

The invention relates to a method for treating cachexia. More particularly, the invention relates to a method of treating cachexia involving the removal or inactivation of macrophage inhibitory cytokine-1 (MIC-1) present in the blood, plasma or serum of a cachexia patient.

### BACKGROUND TO THE INVENTION

Normal weight control is important to good health and wellbeing. Obesity, in particular, may greatly increase morbidity and mortality in subjects, however lower than average weight can also be problematic. Indeed, the condition known as cachexia, which is typically characterised by loss of weight, muscle atrophy, fatigue, weakness and significant loss of appetite, can greatly contribute to morbidity of patients suffering from some chronic diseases (eg cancer, chronic renal disease, chronic inflammatory disease and the eating disorder known as anorexia nervosa). For example, in late stage cancer, cachexia is common (occurring in most terminally ill cancer patients), and is responsible for about a quarter of all cancer-related deaths.

Unfortunately, the control of body weight is a complex process that is, at present, incompletely understood. It is however, known that the process is multifactorial and is influenced by, inter alia, appetite, food ingestion, conversion of food to energy, energy utilisation and expenditure. Further, it has been recognised that there are a number of soluble mediators involved in regulating various aspects of the process including hormones and cytokines such as leptin, ghrelin, melanocortin, agouti-related peptide, and neuropeptide Y (NPY). In work leading to the present invention, the present applicant found that the expression of human TGF-β superfamily cytokine known as macrophage inhibitory cytokine-1 (MIC-1)¹⁻⁷, which is generally expressed in the body at a low level, is dramatically increased in epithelial malignancy, inflammation and injury⁷⁻¹¹ leading to elevated serum MIC-1 levels.

The present applicant has now found that elevated serum levels of MIC-1 in patients with, for example, a late stage epithelial cancer or chronic renal disease, correlate with serum levels observed in transgenic mice over-expressing MIC-1 and showing marked weight loss. It was therefore proposed that cachexia in patients with chronic disease associated with increased MIC-1 expression, is due to the over-expression or decreased clearance of MIC-1 and that by removing or inactivating the MIC-1 contained in the blood, plasma or serum of such patients (eg by using anti-MIC-1 antibodies), it would be possible to reverse or reduce the severity of the weight loss.

### SUMMARY OF THE INVENTION

Thus, in a first aspect, the present invention provides a method of treating or preventing cachexia comprising subjecting blood (eg whole blood), plasma or serum of a subject exhibiting cachexia or prone to developing cachexia to ex vivo treatment so as to remove or inactivate macrophage inhibitory cytokine-1 (MIC-1) present in said blood, plasma or serum and, thereafter, returning the treated blood, plasma or serum to said subject.

In a preferred embodiment, the present invention consists in a method of treating or preventing cachexia comprising the steps of:
(i) providing a suitable substrate for binding MIC-1;
(ii) treating blood, plasma or serum removed from a subject by contacting the blood, plasma or serum ex vivo with said substrate such that MIC-1 present in the blood, plasma or serum is bound to the substrate;
(iii) separating the treated blood, plasma or serum from the substrate; and thereafter
(iv) returning the treated blood, plasma or serum to the subject.

In a second aspect, the present invention provides a method of diagnosing or prognosing cachexia in a subject, said method comprising determining the amount of MIC-1 (particularly, the serum MIC-1 level) present in said subject.

### BRIEF DESCRIPTION OF THE FIGURES

FIG. 1 provides a schematic diagram of the processing of the MIC-1 precursor through to its mature, 112 amino acid form. Cleavage of the propeptide from the mature domain occurs at Arg¹⁹⁶.

FIG. 2 graphically shows the relationship between nude mouse weight and human MIC-1 serum levels in blood collected when the largest of the mouse tumours has reached about 1 cm diameter. Nude mice were xenografted with human DU145 cells engineered to over express either;

(i) full length human MIC-1 (including the propeptide) (series 3),
(ii) mature human MIC-1 (no propeptide) (series 1),
(iii) human MIC-1 including the propeptide but having the furin-like proconvertase site deleted (FURIN DEL) (series 2), and
(iv) vector only negative control (series 4);

FIG. 3 graphically shows the relationship between nude mouse percentage weight loss (compared to weight at the start of the experiment) and human MIC-1 serum levels in blood collected when the largest of the mouse tumours had reached about 1 cm diameter. Nude mice were xenografted with human DU145 cells engineered to over express;

(i) full length human MIC-1 (including the propeptide) (series 3),
(ii) mature human MIC-1 (no propeptide) (series 1),
(iii) human MIC-1 including the propeptide but having the furin-like proconvertase site deleted (FURIN DEL) (series 2), and
(iv) vector only negative control (series 4);

FIG. 4 provides graphical results of the effect of sheep antihuman MIC-1 antibodies on mouse weight (g). (A) On day 27, two mice were given 10 mg (intraperitoneally) of purified IgG from sheep immunised with highly purified recombinant MIC-1 to develop high titre antibodies to human MIC-1. (B) On day 27, two mice were give 10 mg (intraperitoneally) of control purified IgG from normal sheep serum. The graphs A and B show representative data from one of each of the mice in the two groups;

FIG. 5 provides the results of a weight loss assessment with a MIC-1 over-expressing transgenic (TG) mouse line min 28. Body weight was significantly reduced (P<0.001) in both male and female min 28 mice compared to congenic wild type litter mates (3 litters, 59 to 61 days of age);

FIG. 6 provides the results of a weight loss assessment with a MIC-1 over-expressing transgenic (TG) mouse line min 75. Body weight was significantly reduced (P<0.001) in both male and female min 75 mice compared to congenic wild type (WT) litter mates (3 litters, 59 to 61 days of age);

FIG. 7 shows a comparison of body weight (g), of wild type mice (filled symbols, WT) and heterozygous transgenic litter mate mice (TG, open symbols) from seven litters. The number indicates the average weight of heterozygous mice compared to their wild type litter mates within each litter. Newborn WT and TG mice (less than mice <48 h old) are not significantly different in bodyweights;

FIG. 8 shows that administration of a monoclonal antibody (MAb26) to human MIC-1 can reverse the weight loss in nude mice xenografted with human DU145 cells which have been transduced to over-express MIC-1 using a construct of mature human MIC-1 (no propeptide). Mice injected with DU145 cells over expressing MIC-1 started to lose weight rapidly. Administration of a single injection of MAb26, in amounts between 0.1 and 1 mg, at day 11, caused in increase in weight, the magnitude of which, and the duration of which, increased with increasing amounts of MAb26 (A-C). There was no effect of MAb26 on tumour growth (D-F). Untreated mice (G) and mice treated with PBS buffer alone (H) rapidly and continuously lost weight over the course of the experiment. Weight (g) is on the vertical axis;

FIG. 9 shows a comparison of food intake, daily over 3 successive days, in nude mice xenografted with human DU145 cells which have been transduced to over-express MIC-1 using a construct of mature human MIC-1 (no propeptide) and control mice receiving DU145 cells transduced with a control construct;

FIG. 10 shows a comparison of fat pad and muscle weights in nude mice xenografted with human DU145 cells which have been transduced to over-express MIC-1 using a construct of mature human MIC-1 (no propeptide) and control mice receiving DU145 cells transduced with a control construct. MIC-1 bearing DU145 expression tumours are represented by solid bars and the open bars represent mice bearing control tumours. Statistical comparison was undertaken using T test and the number of stars indicates increasing statistical significance from p=0.003 to p<0.0001. There was a marked decrease in the weight of body fat in inguinal fat, epididimal fat and retroperitoneal fat. There was no significant difference in the muscle weight between the two groups of mice. NS=not significant **p<0.01 ***p<0.001;

FIG. 11 shows food intake in MIC-1 transgenic mice compared to wild type controls. 5 wild type (WT) and 6 transgenic (TG) mice were individually housed in cages, and left for 48 hours to adjust to the single housing. Food placed in the hopper was weighed at time point zero. Every 24 hours, food consumed was estimated by subtracting the refusal and the spillage from the weight of the food put into the hopper. Food intake was measured over four, separate 24 hour periods. Food intake per mouse/day was significantly greater in WT animals (p<0.04) (A). However, this difference disappeared when the food intake was corrected for the body weight of the mouse (B);

FIG. 12 shows the weights of organs from MIC-1 transgenic (TG) mice and wild type (WT) mice. Abbreviations: m=male, f=female, epid=epidydimal, ut=uterine, retroperit=retroperitoneal. **p<0.01 ***p<0.001;

FIG. 13 shows the results of assays for MICA binding to fetuin. Purified recombinant MIC-1 (in 0.1% BSA) was incubated with fetuin-coated agarose beads. The beads were then washed and bound material analysed by SDS-PAGE followed by Western blotting with anti-MIC-1 antibody. Lane 1, purified recombinant MIC-1; lane 2, MIC-1 bound to fetuin beads; lane 3, fetuin beads only; lane 4, MIC-1 incubated with agarose beads only. The arrow indicates the MIC-1 bands; and

FIG. 14 shows sections of normal adult mouse brain in the region of the hypothalamus and the third ventricle (V3) were cut and subjected to (A) in situ hybridisation for MIC-1 using ³⁵S-labelled RNA probe and autoradiography and (B) immunohistochemistry using affinity purified polyclonal antibodies to recombinant murine MIC-1. The sections show expression of MIC-1 mRNA and proteins in the region of the arcuate nucleus (AN) and paraventricular region.

### DETAILED DESCRIPTION OF THE INVENTION

It has been previously found that many cancers, especially of epithelial origin, over-express MIC-1 and that serum MIC-1 levels rise in patients with these cancers in proportion to the stage and extent of the disease. Especially in late stages of cancer, these serum levels can reach 3.7 to 50 ng/ml or more, levels which in mice are associated with marked weight loss. By reducing MIC-1 levels or the activity of MIC-1 in cancer patients and other patients exhibiting cachexia or who might be prone to cachexia, it is expected that weight loss, and the subsequent ill-effects on patient well-being and esteem, may be reversed or reduced. In turn, this may assist in the patient's capacity to be treated for the underlying chronic disease (eg cancer or chronic renal disease) and positively respond to the therapy, and thereby reduce morbidity and mortality.

Thus, in a first aspect, the present invention provides a method of treating or preventing cachexia comprising subjecting blood (eg whole blood), plasma or serum of a subject exhibiting cachexia or prone to developing cachexia to ex vivo treatment so as to remove or inactivate macrophage inhibitory cytokine-1 (MIC-1) present in said blood, plasma or serum and, thereafter, returning the treated blood, plasma or serum to said subject.

The subject may be suffering from chronic disease such as cancer (especially epithelial cancers such as breast cancer, prostate, colonic, rectal, bladder and pancreatic cancer), chronic renal disease, chronic inflammatory disease (eg rheumatoid arthritis and Crohn's disease), chronic obstructive pulmonary disease (COPD), cardiac disease such as congestive heart failure, the eating disorder known as anorexia nervosa, or certain infectious diseases such as tuberculosis, acquired immune deficiency syndrome (AIDS) and malaria. These diseases and disorder are commonly associated with cachexia. The subject may therefore already be exhibiting cachexia or, otherwise, is prone to developing cachexia. Typically, the subject will show elevated MIC-1 levels in their blood, plasma or serum (eg serum levels of 3.7²¹ to 50 ng/ml or 5 to 50 ng/ml (although, for some diseases, serum MIC-1 levels as high as 100 to 200 ng/ml are observed) or, at least, serum levels of MIC-1 that are consistently at the high end of the normal range of serum MIC-1 levels of 0.2 to 1.15 ng/ml²⁰. Such subjects can be selected, if necessary, by detection of an elevated MIC-1 level (eg from a blood, plasma or serum sample), using an assay for MIC-1 (eg a MIC-1 ELISA⁴).

In addition to the treatment of subjects suffering from the diseases and disorder mentioned in the preceding paragraph, the method of the present invention may also be suitable for treating or preventing cachexia associated with conditions or treatments wherein MIC-1 is over-expressed (eg injury, stress, and radiotherapy and chemotherapy) or wherein MIC-1 clearance is reduced.

By treating the subject's blood, plasma or serum ex vivo so as to remove or inactivate MIC-1 and thereafter returning the treated blood, plasma or serum to the subject, the serum MIC-1 level may be effectively reduced which, in turn, may result in increased appetite and/or lead to an increase in body weight or, at least, a reduction in any loss of body weight in the subject. Desirably, the blood, plasma or serum of the subject will be treated such that the level of MIC-1 is, for serum, at the low end of the normal range of serum MIC-1 levels of 0.2 to 1.15 ng/ml or, for blood or plasma, at a level which corresponds to an amount at the low end of the normal range of serum MIC-1 levels of 0.2 to 1.15 ng/ml (nb in plasma, the corresponding amount would be substantially equivalent since the major component of plasma is serum with the difference merely constituting fibrinogen and other clotting factors, while for blood, the corresponding amount would be about twice the amount of that in serum). It may be necessary to treat the subject's blood, plasma or serum regularly in order to maintain a reduced MIC-1 level to achieve a desired outcome (eg increased appetite).

The MIC-1 present in the blood, plasma or serum may be removed or inactivated using, for example, a molecule and/or substrate which binds to MIC-1.

Suitable molecules which bind to MIC-1 include MIC-1 receptors and fragments thereof (eg soluble extra-cytoplasmic receptor domains of MIC-1 receptors), and other soluble molecules or matrix-associated proteins that bind to mature MIC-1. A particular example of a soluble molecule that binds to mature MIC-1 and which is useful in the present invention is fetuin. Fetuin²² is a glycoprotein that is abundant in foetal blood, where it is involved in the transport of a variety of substances. MIC-1-binding fragments of fetuin are also suitable for use in the present invention.

Suitable substrates which may be used to remove or inactivate MIC-1 present in the blood, plasma or serum, may comprise, for example, a natural or synthetic substance, perhaps an extracellular matrix (ECM)-like substance, that binds to mature MIC-1. Such substrates could be provided in the form of a bead, fibre, membrane or other surface which may be readily contacted with blood, plasma or serum to enable the “capture” of MIC-1 (ie binding of MIC-1 to the substrate) and thereby enable the removal of MIC-1 from the treated blood, plasma or serum.

Preferably, the ex vivo treatment of the blood, plasma or serum involves the use of an antibody, or functional fragment thereof (eg Fab fragments or recombinant scFv fragments¹²), which specifically binds to MIC-1 (ie an anti-MIC-1 antibody or fragment thereof). The use of an anti-MIC-1 antibody or fragment thereof (as well as other molecules which bind to MIC-1 such as a MIC-1 receptor or fragment thereof) in accordance with the present invention, preferably involves providing that MIC-1-binding molecule on the surface of a suitable substrate which may be readily contacted with the blood, plasma or serum to enable the “capture” of MIC-1 (ie binding of MIC-1 to the substrate through the said MIC-1 binding molecule) and thereby enable the removal of MIC-1 from the treated blood, plasma or serum. The substrate, in this case, may comprise an inert bead (eg polystyrene or agarose bead), fibre, membrane (eg a high flux membrane such as a polyacrylonitrile or polysulphone membrane) or other surface. Preferably, the MIC-1-binding molecule is bound to the substrate surface by covalent linkage, however other forms of bonding (eg electrostatic bonding) may also be suitable. Routine methodologies for binding the MIC-1-binding molecule to a substrate surface are well known to persons skilled in the art.

In a preferred embodiment, the present invention consists in a method of treating or preventing cachexia comprising the steps of:
(i) providing a suitable substrate for binding MIC-1 (eg a substrate provided with a MIC-1-binding molecule);
(ii) treating blood, plasma or serum removed from a subject by contacting the blood, plasma or serum ex vivo with said substrate such that MIC-1 present in the blood, plasma or serum is bound to the substrate;
(iii) separating the treated blood, plasma or serum from the substrate; and thereafter
(iv) returning the treated blood, plasma or serum to the subject (eg by infusion).

The method of the preferred embodiment may involve the use or modification of any one or more routine methodologies for extracorporeal treatment of blood, plasma or serum (eg haemodialysis, haemopheresis, apheresis and plasmapheresis methodologies). Thus, for example, where the subject is suffering from chronic renal disease and requires haemodialysis (eg using continuous arteriovenous haemofiltration (CAVH), continuous venovenous haemofiltration CVVH), or slow continuous ultrafiltration (SCUF)), conveniently the haemodialysis may be modified to incorporate the method of the preferred embodiment (ie such that the blood, prior to its return to the subject, is also contacted with the substrate for binding MIC-1, such that MIC-1 present in the blood is removed by becoming bound to the substrate).

The method of the present invention may be used to treat or prevent cachexia in combination with other cachexia therapies or prophylactic treatments such as, for example, enteral and parenteral nutrition. Parenteral nutrition may be conveniently given to the subject by admixing, ex vivo, appropriate nutritive substances to the blood, plasma or serum, either before, during or after the removal or inactivation of MIC-1 in accordance with the present invention.

Further, in a second aspect, the present invention provides a method of diagnosing or prognosing cachexia in a subject, said method comprising determining the amount of MIC-1 (particularly, the serum MIC-1 level) present in said subject.

The method of the second aspect may be used in combination with other cachexia assays involving, for example, observation and/or measurement of weight loss, detection of cachexia markers (eg cachexia-associated levels of IL-6 or ghrelin in blood, plasma or serum).

Preferably, the method will involve determining whether the subject shows an elevated MIC-1 level associated with cachexia (eg serum levels of 3.7 to 50 ng/ml or more, or at least, serum levels of MIC-1 that are consistently at the high end of the normal range of serum MIC-1 levels of 0.2 to 1.2 ng/ml). Such a determination may be made using an assay for MIC-1 (eg a MIC-1 ELISA⁴). The determination of an elevated MIC-1 level should also indicate that the cachexia of the particular patient will be treatable/preventable through use of the method of the first aspect of the present invention or, otherwise, by administering to the subject a MIC-1 inhibiting agent in accordance with, for example, those agents and methods described in the present applicant's co-pending International patent application No PCT/AU2005/000525 (WO 2005/099746). Preferred MIC-1 inhibiting agents include those MIC-1-binding molecules mentioned above.

In order that the nature of the present invention may be more clearly understood, preferred forms thereof will now be described with reference to the following non-limiting examples.

### EXAMPLES

### Example 1

### Regulation of Serum MIC-1 Levels

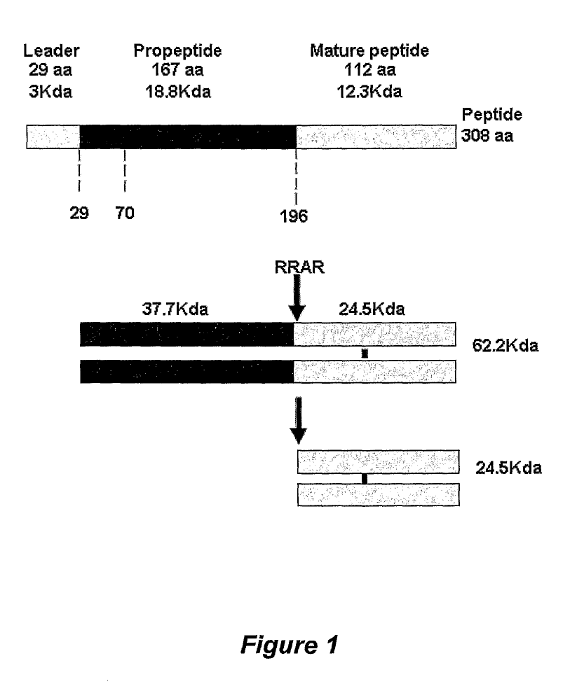

MIC-1, like other members of the TGF-β superfamily of proteins, is synthesised as a precursor containing an N-terminal propeptide and a C-terminal mature MIC-1 domain. The precursor undergoes disulphide-linked dimerisation in the endoplasmic reticulum (ER) and, once dimerised, leaves the ER for the Golgi apparatus, where a furin-like convertase cleaves it at a conserved RXXR site (amino acid 196) (SEQ ID NO: 1). This cleavage removes the propeptide from the mature C-terminal domain and MIC-1 is thus released as a 24.5 kJ) disulphide linked dimer¹ of the mature 112 amino acid polypeptide (FIG. 1).

It has been previously found that substantial amounts of MIC-1 are normally secreted in an unprocessed form. For example, it has been found that endogenous unprocessed proMIC-1 is secreted from a variety of cells including the trophoblast cell line BeWo⁴, the prostate cancer cell lines LnCAP and PC3, the pancreatic cell line Panc 1 and the monocytoid cell line U937. In the prostate adenocarcinoma line, LnCAP, it has been found that unprocessed proMIC-1 associates with extracellular matrix (ECM), whilst mature MIC-1 locates to the conditioned medium¹³. Some preliminary studies with transfected Madin-Darby canine kidney (MDCK) cells, has also demonstrated that ECM association is also mediated by a C-terminal region of the propeptide at amino acids 144-195. Additionally, both purified recombinant propeptide and proMIC-1 interact with heparin through the same C-terminal region of the propeptide.

The association of proMIC-1 with ECM, suggests that ECM association may provide local storage of latent MIC-1, wherein processing of the stored proMIC-1 would result in the rapid release of mature MIC-1 (which has little affinity for ECM) into the circulation. To test this concept, a tumour xenograft model in nude mice¹⁴ was developed.

### Materials and Methods

Using the DU145 human prostate carcinoma line¹⁵, which makes no endogenous MIC-1 (largely because the cells produce no functional p53) and is therefore useful as a vehicle for expressing various human MIC-1 constructs, permanently transfected and subcloned DU145 cell lines were generated which were transduced with eukaryotic expression vectors (IRES II EGFP vector, Clontech Laboratories, Inc., Mountain View, Calif., United States of America) containing sequences encoding either;

(i) full length human proMIC-1 (except using an FSH leader peptide, rather than the natural leader)¹,
(ii) mature human MIC-1 (no propeptide, but including an FSH leader),
(iii) human proMIC-1 (including an FSH leader) with a deletion of the amino acid sequence RGRRRAR (SEQ ID NO: 2) including the furin-like proconvertase site (shown in bold), thereby preventing processing and subsequent release of mature MIC-1 from the propeptide, and
(iv) vector only negative control⁵.

High expressing subclones were selected based on EGFP expression. These cells were injected subcutaneously into the flank of immunodeficient BALM nu/nu nude mice. Mice were monitored regularly and their weight determined on a 2-3 daily basis. Mice were sacrificed about 2 months after injection or when tumour diameter reached 1.1 cm. Serum was obtained from these mice just prior to sacrifice, for estimation of the level of human MIC-1 by ELISA^{4, 14, 16}. This ELISA for human MIC-1 does not cross react with murine MIC-1, and has been previously used for the successful and exclusive measurement of human tumour MIC-1 levels in mice¹⁴.

### Results

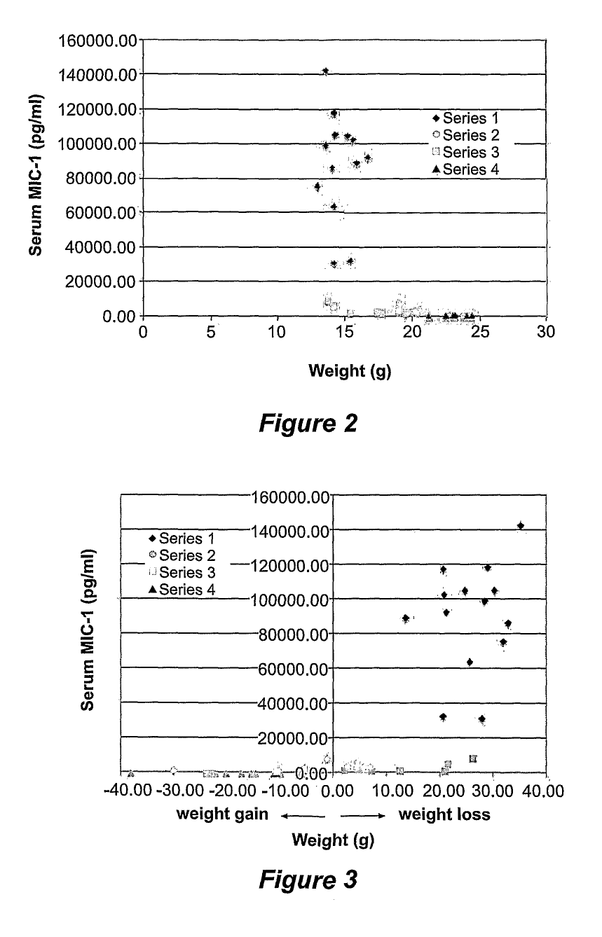

The results are shown in FIGS. 2 and 3. Tumour mice expressing mature MIC-1 showed a dramatically elevated level of serum MIC-1. In contrast, mouse tumours expressing the FURIN DEL mutant of MIC-1, which could not be processed normally and therefore contained the propeptide, had markedly lower serum MIC-1 levels. By extrapolation from in vitro and in vivo data, it appears that this result is due to tight association of the FURIN DEL mutant with the ECM.

### Discussion

The results obtained in this example indicate that the MIC-1 propeptide is important in regulating the distribution of MIC-1 between tissues and blood. As such, any substances that bind to the MIC-1 propeptide (eg heparin and heparan sulphate), or otherwise compete with matrix binding sites on the propeptide (eg recombinant purified propeptide itself) would be expected to increase the level of MIC-1 in the circulation. As a consequence, functions mediated by serum MIC-1 would be modulated.

### Example 2

### Modulation of Appetite by MIC-1

Over the course of the investigation described in Example 1, it was noted that of the xenograft model mice, those bearing a tumour over-expressing MIC-1, either lost weight, or did not gain as much weight as control mice. Studies were therefore conducted to determine the extent and reason for the observed effect on mice weight.

### Materials and Methods

The mice were weighed just before sacrifice and weight/% weight loss compared against the measured serum MICA levels (ie as determined by ELISA described in Example 1).

To assess whether serum MIC-1 levels were responsible for observed weight loss, a second study was conducted wherein nude mice were injected subcutaneously with the DU145 clone over expressing mature human MIC-1 (which had previously been associated with the highest serum MIC-1 levels) and at day 27, after the mice had lost substantial weight, injected intraperitoneally with either 1 mg or 10 mg of control purified sheep IgG or IgG purified from serum from sheep that had been immunised with recombinant human MIC-1 and had high titre antibodies to human MIC-1. This sheep anti-human MIC-1 IgG reacted with high affinity to human MIC-1 and had been previously used in a MIC-1 ELISA.

To further demonstrate that the observed weight loss was mediated by MIC-1 and not another tumour-derived product, an evaluation of weight loss was made of two transgenic mouse lines (min 28 and min 75; both created in C57B16 mice) which over express murine MIC-1 under the control of the macrophage specific c-fms promoter.

### Results

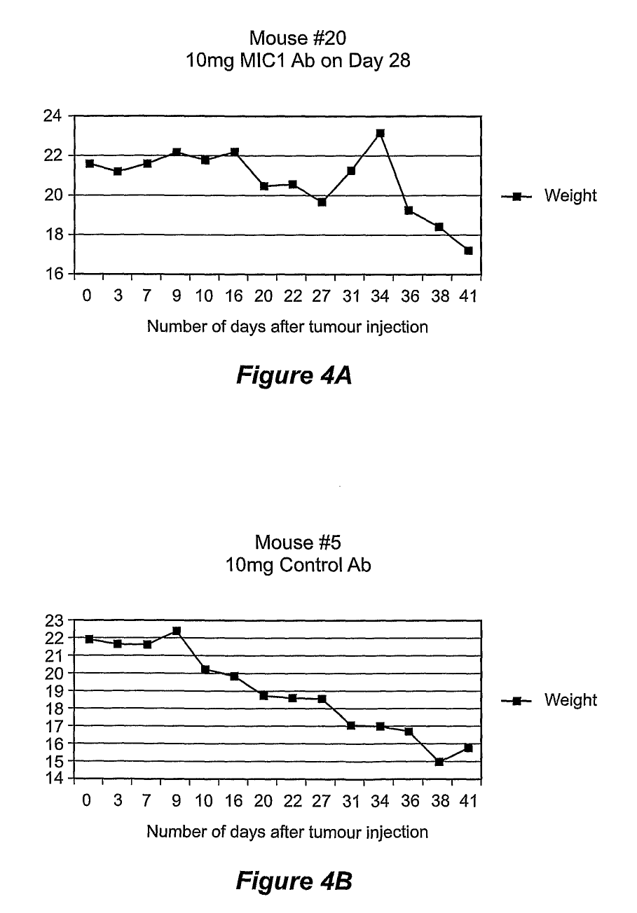

In the studies conducted with sheep anti-human MIC-1 IgG, it was found that 1 mg of sheep anti-human MIC-1 IgG made no difference to the weight of the mouse (data not shown), however 10 mg of anti-MIC-1 IgG (see FIG. 4A) induced a rapid weight gain in the respective tumour-bearing nude mice (cf. the results shown in FIG. 4B with 10 mg of control IgG). This weight gain peaked 5 to 6 days after administration of the antibodies, and then gradually the mice began to lose weight over the following 7 to 10 days.

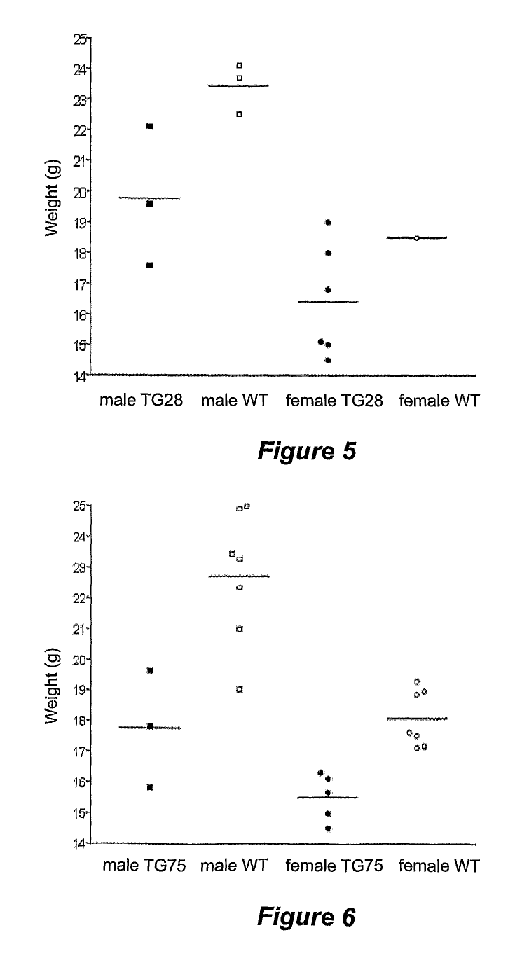

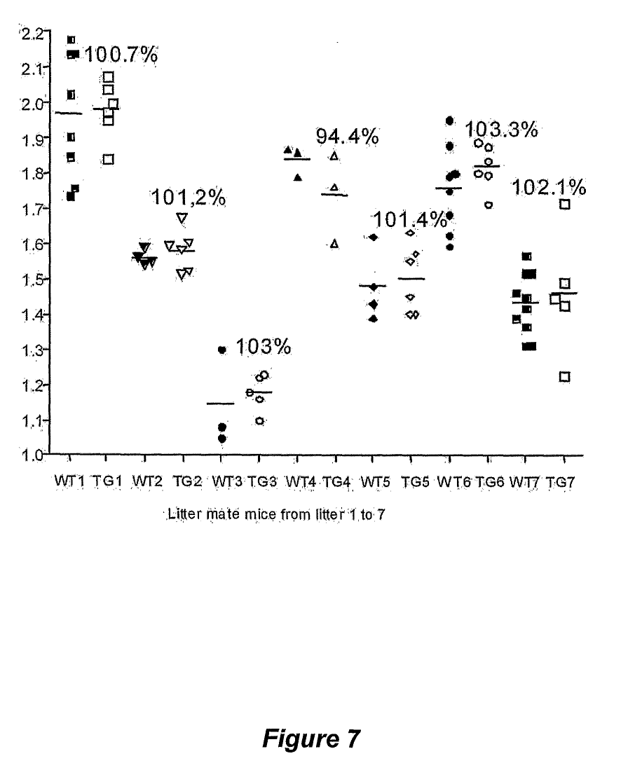

The results of the weight loss assessment in the transgenic mice lines min 28 and min 75 are shown in FIGS. 5 to 7 and indicate that these mice are also substantially smaller than their wild type congenic littermates. In these mice, weight at birth is equal and differences in weight start appearing after the first few weeks of life.

### Discussion

The observed weight loss was very dramatic in some mice and was found to be related to the serum level of tumour-derived human MIC-1. The mice transduced with a DU145 clone over expressing mature human MIC-1 had by far the highest levels of serum MIC-1 and these mice lost weight at a dramatic rate. Observation of animal behaviour, indicated that a major reason for this, was a dramatic reduction in food ingestion by these mice. The finding that the weight loss could be reversed by administration with sheep anti-MIC-1 IgG (but not control IgG) demonstrates that the weight loss was due to MIC-1. This was corroborated by the weight loss assessment with the transgenic mice lines min 28 and min 75. In these mice, which have markedly elevated serum MIC-1 levels even though MIC-1 expression is macrophage-specific, a significant weight differential was observed as compared to congenic wild type mice. This weight loss effect occurred after birth, since both the transgenic mice lines and their congenic wild type litter mates had identical birth weights (ie as measured 24 hours after birth).

### Example 3

### Weight Loss Associated with MIC-1 Secreting Tumour is Reversed by Administration of an anti-MIC-1 Monoclonal Antibody

### Results and Discussion

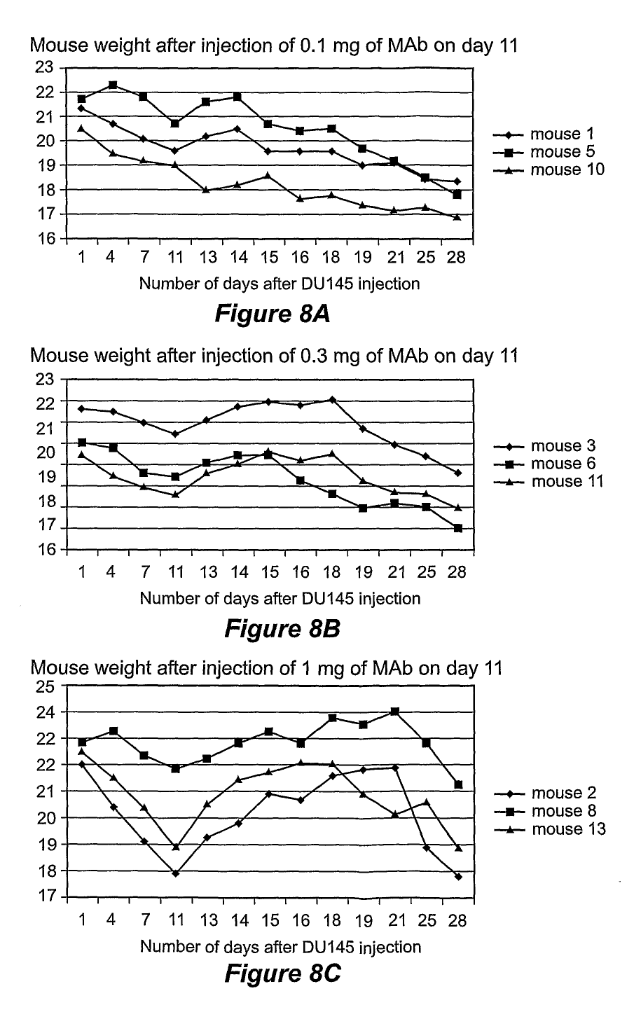

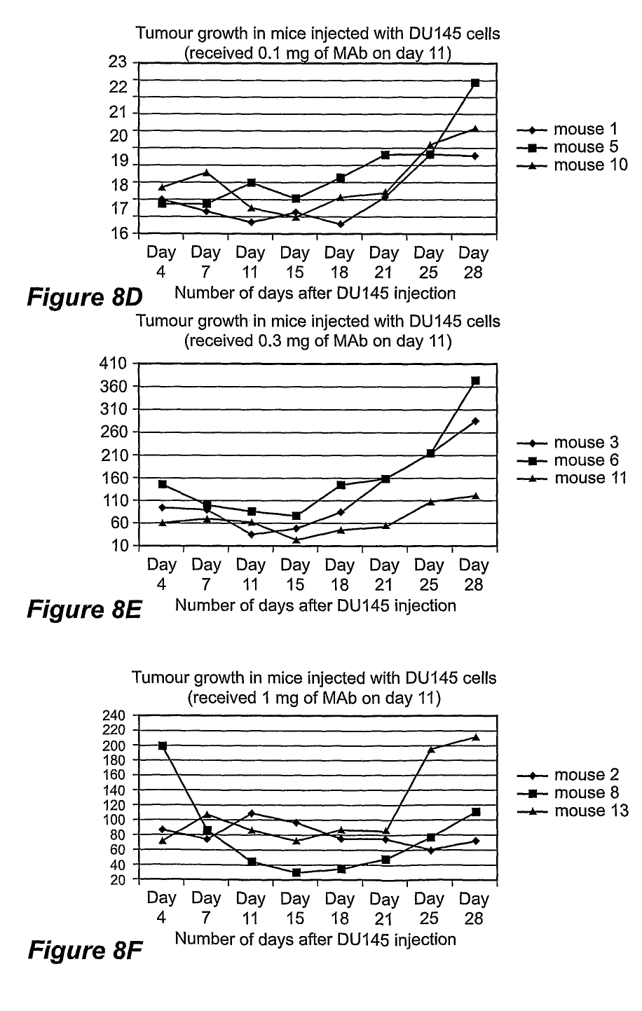

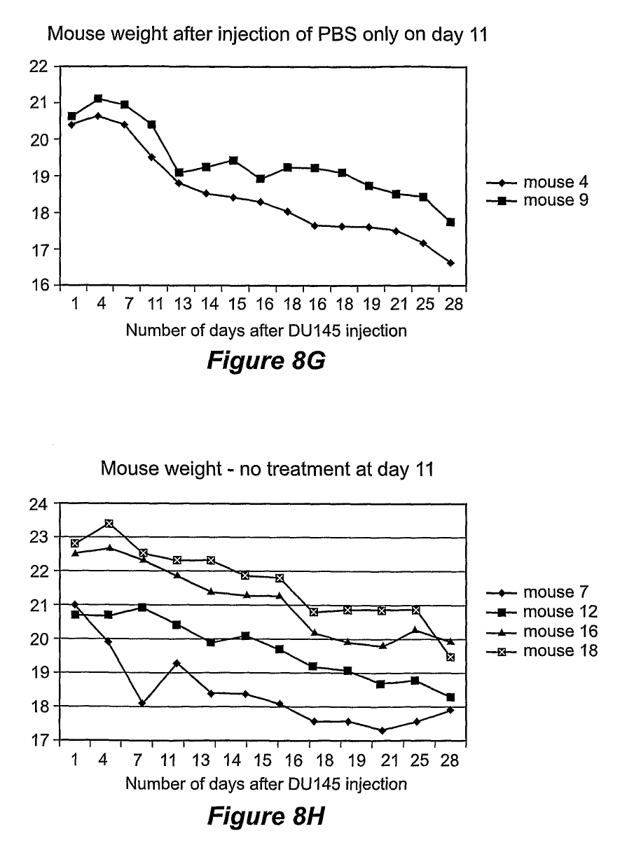

The xenograft mouse model was established in nude mice (as described above) into whose flanks were injected either DU145 cells engineered to over-express mature MIC-1. Mice injected with DU145 cells over expressing MIC-1 started to lose weight rapidly. Administration of a single injection of a monoclonal antibody to MIC-1 (MAb26), in amounts between 0.1 and 1 mg, at day 11, caused an increase in weight, the magnitude of which, and the duration of which increased with increasing amounts of MAb26 (FIG. 8A-C). At the highest dose of approximately 1 mg, the weight had risen to the pre-xenograft level and took approximately 17 days to decrease again to the same weight as when the antibody was first administered. There was no effect of MAb26 on tumour growth (FIG. 8D-F) and untreated mice (FIG. 8G) and mice treated with phosphate buffered saline (FIG. 8H) (PBS) alone, rapidly and continuously lost weight over the duration of the experiment.

### Example 4

### Effect on Food Intake in Mouse Xenograft Model

### Materials and Methods

A xenograft model was established in nude mice (as described above) into whose flanks were injected either DU145 cells engineered to over-express mature MIC-1, or bear a control plasmid. On day 8 after injection of the DU145 cells over-expressing MIC-1, when the average tumour volume was 56 mm³ and the average weight loss 7%, food intake was measured for 3 consecutive 24 hour time periods. The mice were left in groups of 5 per cage. Food placed into the hopper and litter were weighed at time point 0. After 24 hours, food consumed was estimated by subtracting refusal and spillage from food put into the hopper. Food intake for the control mice was measured in the same way, but on day 21 after tumour injection when the tumour volume had reached an average of 70 mm³.

### Results

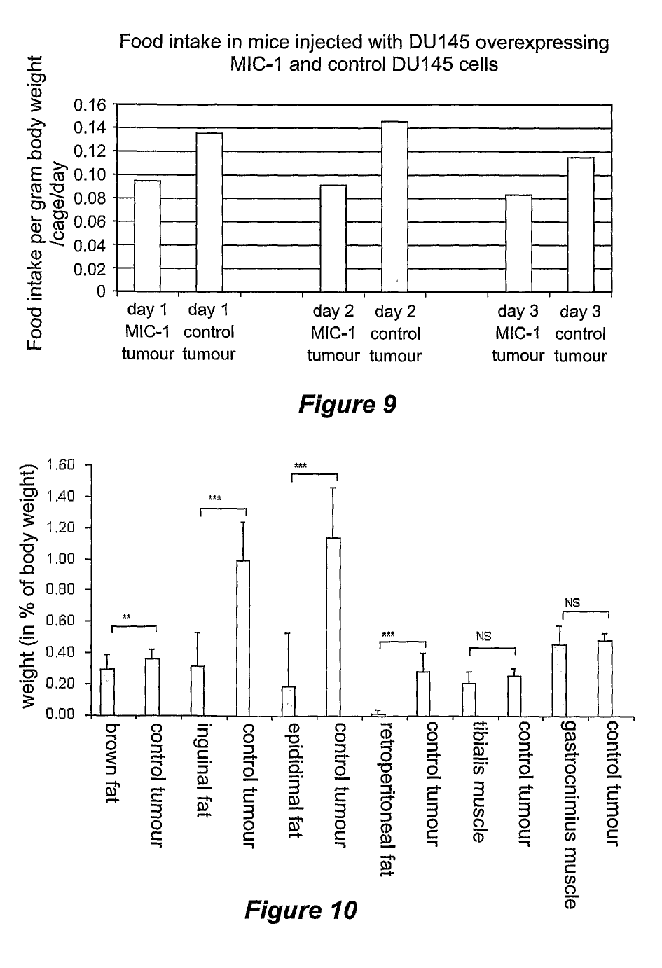

Mice injected with DU145 over-expressing MIC-1 ate significantly less food (about 30%) on day 1, 2 and 3 (p=0.01, 0.0001 and 0.02) than the control mice (FIG. 9). A direct measurement of fat mass in these mice indicated that MIC-1 over-expression was associated with a marked reduction in fat mass in the epididymal, inguinal, and retroperitoneal areas with no reduction in mass in two representative muscles (FIG. 10).

### Example 5

### Measurement of Serum Metabolic Markers in Mouse Xenograft Model

### Materials and Methods

A xenograft model was established in nude mice (as described above) into whose flanks were injected either DU145 cells engineered to over-express MIC-1 or control DU145 cells. At 11-16 days after injection of the DU145 tumour cells over-expressing MIC-1 and 21-30 days after injection of the control tumour, when tumour volumes had reached 100-200 mm³, and/or the mice had lost approximately 18% body weight, the mice were sacrificed. From previous experiments, it was known that serum levels of tumour derived human MIC-1 were between 15 and 58 ng/ml. Serum was collected by cardiac puncture and assayed for the metabolic markers using commercial immunoassays. Statistical comparison was undertaken using the student T test.

### Results and Discussion

Measurement of a range of metabolic markers in mice demonstrated a statistically significant reduction in MIC-1 over-expressing tumor mice of triglyceride and free fatty acids as well as glucagon and IGF-1 (data not shown). There was also a reduction in leptin levels that is consistent with reduction in fat mass, an indication that it is very unlikely that MIC-1 reduced food intake is mediated by MIC-1 stimulation of leptin. The difference for glucose was just short of statistical significance at p=0.053. These finding are largely in keeping with starvation and loss of fat mass.

### Example 6

### Measurement of Fat Pad and Muscle Weight in Mouse Xenograft Model

### Materials and Methods

A xenograft model was established in male nude mice (as described above). Into the flanks of 20 mice were injected DU145 cells engineered to over-express MIC-1 and into 20 mice were injected DU145 cells transduced with a control plasmid. At 11-16 days after injection of the DU145 tumour cells over-expressing MIC-1 and 21-30 days after injection of the control tumour, when tumour volumes had reached 100-200 mm³, and/or the mice had lost approximately 18% body weight, the mice were sacrificed. Interscapular brown adipose tissue, inguinal, epididymal, and retroperitoneal fat and also tibialis and gastrocnemius muscle carefully dissected, removed and weighed and the weight was corrected for body weight.

### Results and Discussion

There was no reduction in brown fat but there was a marked decrease in the weight of body fat (ie white fat) in inguinal fat, epididymal fat and retroperitoneal fat (FIG. 10). There was no significant difference in the muscle weight between the two groups of mice (FIG. 10). However, using more sensitive total lean body mass analysis using the PIXImus imager (GE Lunar) indicated that there was an overall reduction in lean body mass. It also confirmed a much greater reduction in total fat mass and abdominal fat mass.

### Example 7

### MIC-1 Transgenic Mice

### Results and Discussion

Transgenic mice were engineered to over-express MIC-1 from monocytoid cells under the control of the c-fms promoter. These mice have systemically elevated MIC-1 levels, appear well and breed normally. They are indistinguishable from wild type mice but do show a significant growth retardation starting at about 3 weeks and into adulthood (FIG. 5-7). This effect was observed in two independent transgenic lines called min 75 and min 28.

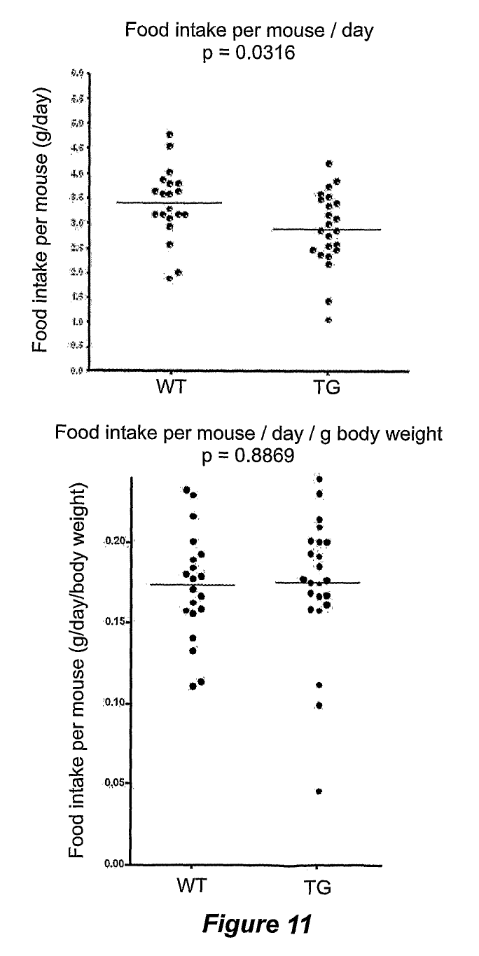

Like the tumour xenograft mice, the MIC-1 over-expressing transgenic mice ate significantly less than their wild type counterparts, but this difference disappears if the food intake is corrected for mouse weight (FIG. 11). It is believed that increased MIC-1 levels from birth result in decreased food intake which results in decreased size and the reach an equilibrium in which their size is appropriate for their reduced food intake. Measurement of the same metabolic markers in the transgenic animals, as in the tumour xenografted mice only showed a significant difference in IGF-1 levels, which are reduced in the MIC-1 transgenic mice.

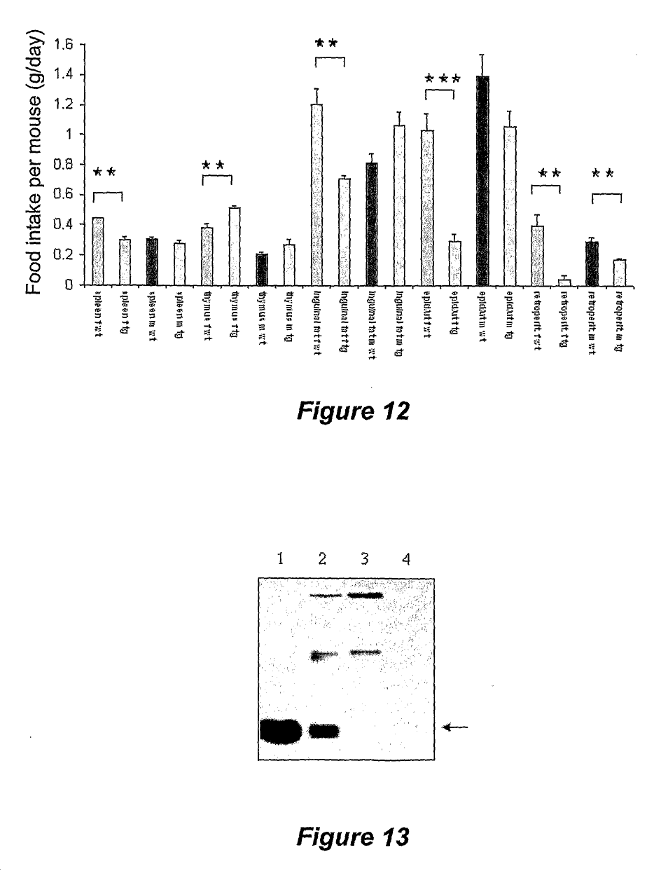

Measurement of fat mass in inguinal, epididymal/uterine and retroperitoneal areas showed a decreased fat mass in the over expressing transgenic mice that was more prominent in female compared to male mice (FIG. 12). Beside a smaller spleen and a larger thymus, all three analysed fat pads were reduced in size. In absolute terms, there was no difference between the weights of WT versus TG thymus.

### Example 8

### Control of Serum MIC-1 Levels by Fetuin

The presence of serum MIC-1, at a mean concentration of 450 pg/ml in all subjects, suggests that like some other TGF-β superfamily cytokines, MIC-1 may bind to one or more circulating modulators. The glycoprotein, fetuin is widely expressed in cells and tissues and is present in blood serum. The following investigation was made to determine whether MIC-1 may interact with this glycoprotein.

### Materials and Methods

Purified recombinant, mature MIC-1 (in 0.1% BSA) was incubated with fetuin-coated agarose beads. The beads were then washed and bound material analysed by SDS-PAGE followed by Western blotting with anti-MIC-1 antibody: Lane 1, purified recombinant MIC-1; Lane 2, MIC-1 bound to fetuin beads; Lane 3, fetuin beads only; Lane 4, MIC-1 incubated with agarose beads only.

### Results and Discussion

The results, shown in FIG. 13, clearly indicate that mature MIC-1 interacts and binds with fetuin. Fetuin thereby offers an alternative to the use of anti-MIC-1 antibodies for removing MIC-1 from a patient's blood to modulate body weight control.

### Example 9

### Analysis of MIC-1 Expression in Normal Mouse Brain

### Results and Discussion

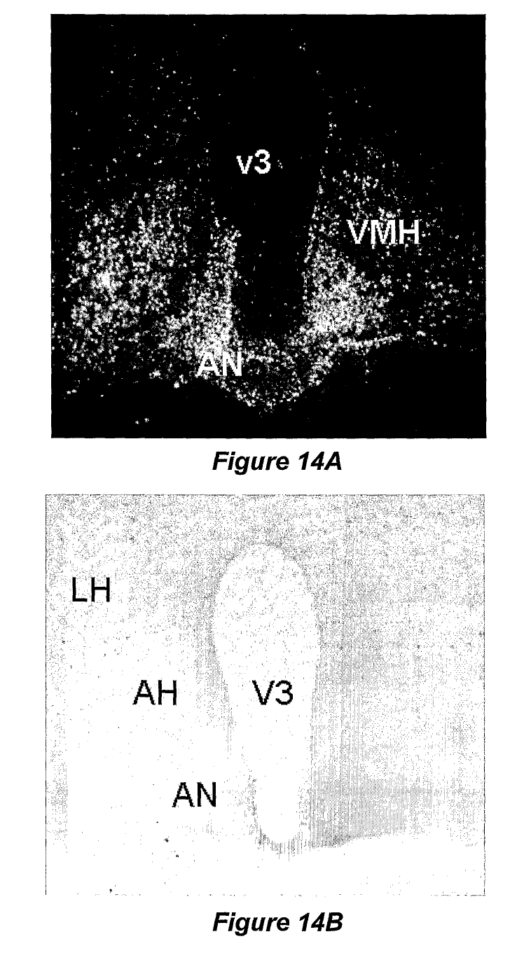

Food intake and appetite are controlled by a complex array of mechanisms, many of which are located within the central nervous system. The area within the nervous system controlling many basal bodily functions such as appetite and body temperature are localised within the area of the hypothalamus. In the case of appetite, many of the complex factors regulating this process are localised to the arcuate nucleus of the hypothalamus and many of the mediators and receptors for mediators such as neuropeptide Y are localised in this area. The blood brain barrier in this area is also leaky and it is one of the very limited areas of the brain where there is an opportunity for systemic molecules to cross the blood brain barrier and act directly in the brain. It is considered that MIC-1 is able to exert a direct effect on the acuate nucleus and hypothalamus by this mechanism. However, MIC-1 is also expressed within this region of the normal mouse brain (FIG. 14). It does not represent diffusion of circulating MIC-1 as indicated by studies of in situ hybridisation which demonstrate co-localisation of MIC-1 mRNA and protein in the area of the acuate nucleus, periventricular area and paraventricular hypothalamus. The localisation of MIC-1 in those areas of normal brain, strongly associated with functions such as appetite control, provides a strong argument for the role of MIC-1, both from the peripheral circulation, and endogenously produced within the brain, in controlling this important function.

### Example 10

### Serum MIC-1 Levels Correlate with the Degree of Weight Loss in Patients with Advanced Prostate Cancer

### Results and Discussion

To determine the relevance of MIC-1 to cachexia in humans, serum MIC-1 levels were measured in patients recruited into a well-characterised cohort of patients with advanced prostate cancer (PCa), in which serum IL-6 levels had been associated with cachexia¹⁷. Serum MIC-1 levels were significantly elevated in patients with advanced cancer with cachexia compared to those with advanced cancer without cachexia (12416±10235 pg/ml vs 3265±6370 pg/ml (mean±SD); p<0.0001; Mann-Whitney-U test). Similarly, serum levels of IL-6 were elevated in cachexia patients (33.8±64.2 pg/ml vs 7.8±3.4 pg/ml, p<0.002; Mann-Whitney-U test). Also, serum MIC-1 was weakly but significantly and positively correlated with serum IL-6 levels (r=0.2949, p<0.04; linear regression). Additionally, in single and multivariate logistic regression, both serum MIC-1 and IL-6 levels were independent predictors of the presence of cancer cachexia (p=0.0002, p<0.0001, respectively; univariate logistic regression: p=0.0017, p=0.0005, respectively; multivariate logistic regression). However, the best objective, quantifiable measure of cachexia is weight loss. Serum MIC-1 levels were significantly associated with the degree of prostate cancer associated weight loss (p=0.0002, r=0.4899; linear regression), while there was no such relationship for serum IL-6 (p=0.6303; linear regression).

### Example 11

### Serum MIC-1 Levels are Related to BMI in Patients with Chronic Renal Failure

### Results and Discussion

Chronic renal failure, like advanced cancer is also commonly associated with weight loss and cachexia. Markers of this process such as anorexia, weight loss and BMI are strong predictors of mortality in end stage renal failure¹⁸. As BMI is such a strong predictor of mortality, and elevated serum MIC-1 levels were associated with similar changes in animals, the relationship between serum MIC-1 level and BMI in end stage renal failure was investigated. To do this, serum samples were examined from a cohort of 381 patients with end stage renal failure, that had been not been assembled to study cachexia or other metabolic processes¹⁹.

Patients who died during the study period (up to 3 years) had significantly lower BMI (26.17±5.63; 266 (mean±SD; n), 23.15±4.92; 104: p<0.0001; unpaired t-test). A predialysis serum sample was obtained at study entry and MIC-1 serum level determined. Serum MIC-1 level was correlated with BMI such that increasing serum MIC-1 levels were associated with lower BMI (p=0.0003; r=0.189; linear regression). Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Bootcov M R et al., MIC-1, a novel macrophage inhibitory cytokine, is a divergent member of the TGF-β superfamily cluster. Proc Natl Acad Sci USA 1997 94:11514-11519.
2. Breit S N & M R Bootcov, “Novel TGF-beta like cytokine”, International Patent Application No PCT/AU96/00386 (WO 97/00958).
3. Fairlie W D et al., Expression of a TGF-β superfamily protein, Macrophage Inhibitory Cytokine-1, in the yeast Pichia pastoris. Gene 2000 254:67-76.
4. Moore A G et al., TGF-β superfamily cytokine MIC-1 is present in high concentrations in the serum of pregnant women. J Clin Endocrinol Metab 2000 85:4781-4788.
5. Fairlie W D et al., Epitope mapping of the Transforming Growth Factor-β superfamily protein, MIC-1: Identification of at least five distinct epitope specificities. Biochemistry 2001 40:65-73.
6. Breit S N et al., “Diagnostic assay and method of treatment involving macrophage inhibitory cytokine-1 (MIC-1)”, International Patent Application No PCT/AU01/00456 (WO 01/81928).
7. Fairlie W D et al., MIC-1 is a novel TGF-β superfamily cytokine associated with macrophage activation. J Leukocyte Biol 1999 65:2-5.
8. Koniaris L G, Induction of MIC-1/growth differentiation factor-15 following bile duct injury. J Gastrointest Surg 2003 7(7):901-905.
9. Welsh J B et al., Analysis of Gene Expression Identifies Candidate Markers and Pharmacological Targets in Prostate Cancer. Cancer Res 2001 61:5974-5978.
10. Buckhaults P et al., Secreted and cell surface genes expressed in benign and malignant colorectal tumors. Cancer Res 2001 61:6996-7001.
11. Welsh J B et al., Large-Scale Delineation of Secreted Protein Biomarkers Overexpressed in Cancer Tissue and Serum. Proc Natl Acad Sci USA 2003 100:3410-3415.
12. Pluckthun A, Antibody engineering: advances from the use of Escherichia coli expression systems. Bio/Technology 1991 9:545-551.
13. Bauskin A R et al., The propeptide mediates formation of stromal stores of PROMIC-1: role in determining prostate cancer outcome. Cancer Res 2005 65(6):2330-2336.
14. Brown D A et al., An antibody based approach to high volume genotyping for MIC-1 polymorphism. Biotechniques 2002 33(1):118-120, 122, 124 passim.

15. Liu T et al., MICA reduces cell adhesion and induces apoptosis in prostate cancer cells. Cancer Res 2003 63:5034-5040.
16. Koopmann J et al., Serum macrophage inhibitory cytokine 1 as a marker of pancreatic and other periampullary cancers. Clin Cancer Res 2004 10(7):2386-2392.
17. Pfitzenmaier, J et al., Elevation of cytokine levels in cachectic patients with prostate carcinoma. Cancer 2003 97:1211-1216.
18. Kovesdy, C P et al., Inverse association between lipid levels and mortality in men with chronic kidney disease who are not yet on dialysis: effects of case mix and the malnutrition-inflammation-cachexia syndrome. J Am Soc Nephrol 2007 18:304-311.
19. Apple, F S et al., Predictive value of cardiac troponin I and T for subsequent death in end-stage renal disease. Circulation 2002 106:2941-2945.
20. Brown, D A et al., MIC-1 serum level and genotype: associations with progress and prognosis of colorectal carcinoma. Clin Cancer Res 2003 9:2642-2650.
21. Kempf, T et al., Prognostic utility of growth differentiation factor-15 in patients with chronic heart failure. J Am Coll Cardiol 2007 50(11):1054-1060.
22. Demetriou, M et al., Fetuin/α2-HS glycoprotein is a transforming growth factor-β type II receptor mimic and cytokine antagonist. J Biol Chem 1996 271(22):12755-12761.

1. A method of treating or preventing cachexia comprising subjecting blood, plasma or serum of a subject exhibiting cachexia or prone to developing cachexia to ex vivo treatment so as to remove or inactivate macrophage inhibitory cytokine-1 (MIC-1) present in said blood, plasma or serum and, thereafter, returning the treated blood, plasma or serum to said subject.

2. The method of claim 1, wherein the subject is suffering from cancer, chronic renal disease, or chronic inflammatory disease.

3. The method of claim 1, wherein the subject shows an elevated serum MIC-1 level of 3.7 to 50 ng/ml or more.

4. A method according to claim 1 comprising the steps of:
(i) providing a suitable substrate for binding MIC-1;
(ii) treating blood, plasma or serum removed from a subject by contacting the blood, plasma or serum ex vivo with said substrate such that MIC-1 present in the blood, plasma or serum is bound to the substrate;
(iii) separating the treated blood, plasma or serum from the substrate; and thereafter
(iv) returning the treated blood, plasma or serum to the subject.

5. The method of claim 1, wherein the MIC-1 present in the blood, plasma or serum is removed or inactivated using a MIC-1 binding molecule.

6. A method according to claim 5 comprising the steps of:
(i) providing a MIC-1-binding molecule bound to a suitable substrate;
(ii) treating blood, plasma or serum removed from a subject by contacting the blood, plasma or serum ex vivo with said substrate-bound MIC-1-binding molecule such that MIC-1 present in the blood, plasma or serum is bound to the substrate via the MIC-1-binding molecule;
(iii) separating the treated blood, plasma or serum from the substrate; and thereafter
(iv) returning the treated blood, plasma or serum to the subject.

7. The method of claim 5, wherein the MIC-1 binding molecule is an anti-MIC-1 antibody or functional fragment thereof.

8. The method of claim 5, wherein the MIC-1 binding molecule is fetuin or a MIC-1-binding fragment thereof.

9. The method of claim 1, wherein the subject is suffering from chronic renal disease and said method is incorporated into a haemodialysis therapy for said subject.

10. A method of diagnosing or prognosing cachexia in a subject, said method comprising determining the amount of MIC-1 present in said subject.

11. The method of claim 10, wherein the method involves determining whether the subject shows an elevated serum MIC-1 level of 3.7 to 50 ng/ml or more.

12. The method of claim 10, wherein the method identifies cachexia in a subject that is treatable/preventable by the method of any one of claims 1 to 9 or, otherwise, by administering to the subject a MIC-1 inhibiting agent.
